(19) United States
(12) Patent Application Publication
(10) Pub. No.: US 2007/0258909 A1
(43) Pub. Date: Nov. 8, 2007

(54) Method for the Analysis of 1,1,1,2-Tetrafluoroethane

(75) Inventors: Yves Mahaut, Sint-Pieters-Leeuw (BE); Roland Klug, Idstein (DE)

Correspondence Address: CONNOLLY BOVE LODGE & HUTZ, LLP, P O BOX 2207, WILMINGTON, DE, 19899, US

(73) Assignee: SOLVAY S.A., Brussels (BE)

(21) Appl. No.: 10/592,901

(22) PCT Filed: Mar. 16, 2005
(86) PCT No.: PCT/EP05/51206

§ 371 Date: Mar. 16, 2007

Related U.S. Application Data
(60) Provisional application No. 60/553,756, filed on Mar. 17, 2004.

(51) Int. Cl. G01N 30/02; A61K 9/12; C07C 17/38

(52) U.S. Cl. 424/40; 570/177; 73/23.37

(57) ABSTRACT

Method for the analysis of the content of organic impurities in 1,1,1,2-tetrafluoroethane, in which (a) the 1,1,1,2-tetrafluoroethane is subjected to a gas chromatography operation and; (b) an operation is carried out in which the organic impurities are detected by mass spectrometry and wherein said method is carried out using the specific conditions appended hereto and/or said method is carried out making use of any of the quality control test or validation data included in the specification.

The present invention is related to co-pending U.S. application Ser. No. 10/221,014 whose content is incorporated by reference into the present application. The present application claims the benefit of U.S. application Ser. No. 60/553,756, filed Mar. 17, 2004.

The invention relates to a method for the analysis of the content of organic impurities in 1,1,1,2-tetrafluoroethane, in which method
(a) the 1,1,1,2-tetrafluoroethane is subjected to a gas chromatography operation and;
(b) an operation is carried out in which the organic impurities are detected by mass spectrometry, and wherein said method is carried out using the specific conditions appended hereto and/or said method is carried out make use of any of the quality control test data or validation data appended hereto.

The method according to the invention makes it possible, surprisingly, to determine, in a single analytical operation, the nature and the amount of a large number of organic impurities present in 1,1,1,2-tetrafluoroethane. The method according to the invention even makes it possible to carry out a quantitative detection of several organic impurities exhibiting between them the same retention time in the chromatography operation. In a particularly surprising way, the method according to the invention also makes possible the quantitative detection of impurities which exhibit the same retention time in the chromatography operation as the 1,1,1,2-tetrafluoroethane.

The chromatography operation is preferably a gas chromatography operation.

The stationary phase in the chromatography operation is generally nonpolar. A polymer of polysiloxane type is often employed as stationary phase. A stationary phase composed of optionally crosslinked polydimethylsiloxane has given good results. In the case of gas chromatography, good results have been obtained with an Rtx®-1 gas chromatography column sold by Restek Corp.

In an alternative form, the stationary phase exhibits moderate polarity. Such a stationary phase can be composed, for example, of a mixture of nonpolar polymer as described above with a polar polymer. Such polar polymers are chosen, for example, from polymers functionalized by polar groups, in particular from functionalized polyolefins or polyalkylsiloxanes. The polar group can be chosen, for example, from hydroxyl, ether, ester, phenoxy and, preferably, from nitrile. A polysiloxane of general formula 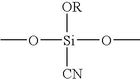
in which R is a C₁ to C₄ alkyl group, preferably a methyl group, is particularly preferred as polar polymer. In the alternative form described above, the content of polar polymer is generally greater than or equal to 1% by weight of the stationary phase. This content is often greater than or equal to 2% by weight. It is preferably greater than or equal to approximately 5% by weight. The content of polar polymer is generally less than or equal to 15% by weight of the stationary phase. The content is often less than or equal to 10% by weight. It is preferably less than or equal to primarily 8% by weight.

The initial faze of the chromatography operation is generally adjusted at the most to 40° C. This temperature is often adjusted at the most to 0° C. This tempera is preferably adjusted at the most to −20° C. Sometimes, this pure is adjusted at the most to 40° C. As a general rule, it is at least −80° C. An initial temperature of about −25° C. is more particularly preferred.

In the chromatography operation, there is generally at least one stage with a constant temperature gradient which provides a controlled temperature rise starting from the initial temperature. This temperature gradient is generally at least 0.1° C./min. It is preferably at least 0.5° C./min.

The temperature gradient is generally at most 50° C. min. It is preferably at most 10° C./min, and more preferably equal to or lower than 4° C.

The column is preferably a capillary column. The length of the column is generally at most 200 n. The length is often at most 120 m. The length of the column is generally at least 20 m.

The injection can be carried out in split or splitless mode. Injection in split mode is preferred.

The carrier gas is often chosen from helium and hydrogen Helium is preferred.

The internal diameter of the column is generally at most 0.32 mm. The diameter is often at most 0.25 mm. The diameter is preferably at most 0.20 mm. The internal diameter of the column is often at least 0.10 mm. The diameter is preferably at least 0.15 mm.

The thickness of the stationary phase film deposited inside the column is generally at least 0.5 μm. The thickness is preferably greater than or equal to approximately 1 μm. The thickness of the stationary phase film deposited inside the column is generally at most 5 μm.

A specific form of the method according to the invention applies preferably when the internal diameter and the thickness of the film lie within the preferred ranges.

The length of the column is, in this specific form, advantageously at least 30 m. In a more particularly preferred way, it is greater than or equal to approximately 40 m. The length of the column is advantageously at most 100 m. In a more particularly preferred way, it is less than or equal to approximately 60 m.

In this alternate form, the term program comprises generally, in addition to the stage carried out at the preferred gradient indicated above, a stage in which the gradient as defined above is generally at least 10° C./min. It is preferably at least 20° C./min. In a more particularly preferred way, the gradient is greater than or equal to approximately 40° C./mL. The temperature gradient in this alternative form is generally at most 50° C./min.

The initial temperature in this alternative form is generally at most −10° C. It is preferably less than or equal to −20° C. The initial temperature in this alternative form is generally at least −50° C.

This alternative form of the method according to the invention makes it possible, surprisingly, to further accelerate the analytical operation while retaining the other advantages of the method according to the invention, in particular with respect to the simultaneous detection and determination of the organic impurities.

Premanufactured gas chromatography columns which make it possible to implement the method according to the invention are available commercially, for example Rtx®-624 from Restec and DB®-624 from J & W.

Detection by mass spectrometry is preferably carried out using the selected ion monitoring (SIM) technique.

According to another preferred alternative form, detection by mass spectrometry is carried out using the time-of-flight (TOF) technique. Mass spectrometers for detection by using the tune-of-flight technique, which are preferred in the method according to the invention, make it possible to record a high number of mass spectra per second, namely approximately 1 to 500, preferably 100 to 500, spectra per second. Spectrometers which can be used for the implementation of the method according to the invention are, for example, those sold by Leco Corporation under the name Pegasus® II and those sold by Thermoquest under the name Tempus™.

The method according to the invention is particularly efficient as determination of the content of all the organic impurities can be obtained by a single analytical operation. That being the case, only this operation has to be validated, that is to say standardized and confirmed. Consequently, the calibration possibly needed between the analysis of various samples is simplified, as shown by the appended validation data.

The method according to the invention makes it possible to achieve a very short duration necessary for the analysis, which can typically be carried out in less than two hours, often in less than one hour. A complete analysis of the impurities can be achieved in a time of approximately 10 minutes. This efficiency makes it possible in particular to improve the performance of industrial manufacturing processes requiring control of the quality of 1,1,1,2-tetrafluoroethane. This is because it is possible to meet, with greater flexibility and speed, urgent orders for 1,1,1,2-tetrafluoroethane and reduce the 1,1,1,2-tetrafluoroethane storage times.

The invention consequently also relates to a process for the manufacture of 1,1,1,2-tetrafluoroethane comprising the use of the analytical method according to the invention for controlling the quality of the 1,1,1,2-tetrafluoroethane.

In an alternative form, the 1,1,1,2-tetrafluoroethane is a purified 1,1,1,2-tetrafluoroethane. In this alternative form the process for the manufacture of 1,1,1,2-tetrafluoroethane often comprises a purification stage. This process preferably comprises
(a) the use of the method according to the invention for the analysis of a crude 1,1,1,2-tetrafluoroethane;
(b) a purification of the crude 1,1,1,2-tetrafluoroethane in order to obtain a purified 1,1,1,2-tetrafluoroethane;
(c) and a second use of the method according to the invention for the analysis of the purified 1,1,1,2-tetrafluoroethane.

If appropriate, the purification can be carried out, for example, according to the production process disclosed in the copending application cited above.

The invention also relates to a process for the manufacture of a pharmaceutical aerosol, comprising at least one 1,1,1,2-tetrafluoroethane of pharmaceutical grade, comprising the use of the analytical method according to the invention for controlling the quality of the 1,1,1,2-tetrafluoroethane of pharmaceutical grade.

The process for the manufacture of a pharmaceutical aerosol according to the invention is particularly suitable for the manufacture of a pharmaceutical aerosol for inhalation comprising at least 1,1,1,2-tetrafluoroethane liquefied under pressure and a medicament. The medicament is preferably present in the form of a powder in the suspended state. The 1,1,1,2-tetrafluoroethane is present as propellent gas.

The process for the manufacture of a pharmaceutical aerosol is particularly advantageous as the analytical method makes it possible to carry out, in a particularly efficient way, the strict quality control laid down for pharmaceutical applications.

The specific conditions and quality control test data are appended hereafter:

| Test method: | Gas chromatography | | (Ph. Eur. 4^{th} Edition 2002, 2.2.28; | |
|---|---|---|---|---|
| | | | Ph. Eur. 4^{th} Edition 2002, 2.2.46) | |
| | Mass spectrometry | | (Ph. Eur. 4^{th} Edition 2002, 2.2.43) | |
| GC Parameter | | | | |
| Apparatus: | Gas chromatograph | | (e.g. Agilent; HP6890) | |
| Column: | Type | | fused silica capillary | |
| | Stationary phase | | 6% cyanopropylphenyl 94% | |
| | | | dimethylpolysiloxane (e.g. J&W DB-624) | |
| | Film thickness | | 1 μm | |
| | Dimension | | 60 m × 0.18 mm | |
| Carrier Gas: | Helium | | (e.g. He 4.6 Messer Griesheim) | |
| | Flow | | 1.2 ml/min (constant flow) | |
| Oven | | Rate | Temperature | Hold time |
| Temperature | Level | [° C./min] | [° C.] | [min] |
| Program: | Beginning | | −25 | 2 |
| | 1 | 2.5 | −12 | 0 |
| | 2 | 4 | 15 | 0 |
| | 3 | 50 | 250 | 2.5 |

| Injector: | Split/splitless with a gas valve system | |
|---|---|---|
| | Temperature | 150° C. |
| | Mode | Split |
| | Split flow | 96 ml/min |
| Gas Valve | Temperature | 150° C. |
| System: | | |
| Injection Volume: | 500 μl (loop) | |
| MS Parameter | | |
| Apparatus: | Time-of-Flight Mass spectrometer e.g. Leco; Pegasus II | |
| Ionization Mode: | EI (70 eV) | |
| Mass Range: | 30 to 325 amu | |
| Acquisition Rate: | 10 spectra/second | |
| Acquisition Time: | 3.5 to 21 min | |
| Temperatures: | Ion Source | 160° C. |
| | Transfer Line | 200° C. |

TABLE 1
Compounds

| | | | Typical | Quantification |
|---|---|---|---|---|
| | | | Retention time | ion |
| No. | Compounds | Abbreviation | [s] | [m/z] |
| 1 | Chlorotrifluoromethane | CFC 13 | 246 | 69 |
| 2 | Trifluoromethane | HFC 23 | 253 | 51 |
| 3 | Chloropentafluoroethane | CFC 115 | 267 | 85 |
| 4 | 1,1,1-Trifluoroethane | HFC 143a | 280 | 65 |
| 5 | Difluoromethane | HFC 32 | 283 | 33 |
| 6 | Trifluoroethene | HFC 1123 | 283 | 82 |
| 7 | Pentafluoroethane | HFC 125 | 288 | 101 |
| 8 | Octafluoro-2-butene (trans) | FC 1318my-trans | 289 | 131 |
| 9 | Octafluoro-2-butene (cis) ¹⁾ | FC 1318my-cis ¹⁾ | 298 | 131 |
| 10 | 2,3,3,3-Tetrafluoropropene | HFC 1234yf | 328 | 114 |
| 11 | 1,1,1,2,2- | HFC 245cb | 329 | 65 |
| | Pentafluoropropane | | | |
| | 1,1,1,2-Tetrafluoroethane | HFC 134a | approx. 340 | main comp. |
| 12 | 1,2,3,3,3- | HFC 1225ye (cis) | 372 | 113 |
| | Pentafluoropropene (cis) | | | |
| 13 | 1,1-Difluoroethane | HFC 152a | 377 | 65 |
| 14 | Fluoroethane | HFC 161 | 377 | 33 |
| 15 | 3,3,3-Trifluoropropene | HFC 1243zf | 378 | 96 |
| 16 | Dichlorodifluoromethane | CFC 12 | 402 | 85 |
| 17 | 1,1,2,2-Tetrafluoroethane | HFC 134 | 404 | 83 |
| 18 | 1,1,1,4,4,4-Hexafluoro-2- | HFC 1336mzz | 437 | 95 |
| | butene (cis) | (cis) | | |
| 19 | Chlorodifluoromethane | HCFC 22 | 444 | 51 |
| 20 | 1,2-Dichloro-1,1,2,2- | CFC 114 | 554 | 100 |
| | tetrafluoroethane | | | |
| 21 | 1,1-Dichloro-1,2,2,2- | CFC 114a | 557 | 103 |
| | tetrafluoroethane | | | |
| 22 | Chloromethane | HCC 40 | 558 | 52 |
| 23 | 1,1-Difluorochloroethene | HCFC 1122 | 575 | 98 |
| 24 | 1-Chloro-1,1,2,2- | HCFC 124a | 588 | 101 |
| | tetrafluoroethane | | | |
| 25 | 1-Chloro-1,2,2,2- | HCFC 124 | 618 | 67 |
| | tetrafluoroethane | | | |
| 26 | 1,2-Difluorochloroethene | HCFC 1122a/1 ²⁾ | 635 | 98 |
| | (isomer 1) ²⁾ | | | |
| 27 | Chlorofluoromethane | HCFC 31 | 671 | 68 |
| 28 | 1,2-Difluorochloroethene | HCFC 1122a/2 ²⁾ | 672 | 98 |
| | (isomer 2) ²⁾ | | | |
| 29 | Chlorobromodifluoro- | CFC 12B1 | 696 | 85 |
| | methane | | | |
| 30 | 1,2-Difluoroethane | HFC 152 | 786 | 33 |
| 31 | 1,1,1-Trifluoro-2- | HCFC 133a | 807 | 118 |
| | chloroethane | | | |
| 32 | 1,1-Dichloro-2,2- | CFC 1112a | 897 | 132 |
| | difluoroethene | | | |
| 33 | Trichlorofluoromethane | CFC 11 | 964 | 101 |
| 34 | 1,2-Dichloro-1,1,2- | HCFC 123a | 1021 | 67 |
| | trifluoroethane | | | |
| 35 | 1,1,1- | HCFC 123 | 1028 | 83 |
| | Trifluorodichloroethane | | | |
| 36 | 1,1,2-Trichloro-1,2,2- | CFC 113 | 1034 | 103 |
| | trifluoroethane | | | |
| 37 | 1,2-Dichlorofluoroethene | HCFC 1121 (trans) | 1055 | 114 |
| | (trans) | | | |
| 38 | 1,2-Dichloro-1,1- | HCFC 132b | 1077 | 99 |
| | difluoroethane | | | |

¹⁾ Because its reference substance is not commercially available, the quantification and validation of the determination of cis-octafluoro-2-butene (FC 1318my/c) is performed using trans-octafluoro-2-butene (FC 1318my/t),
²⁾ Because their isolated reference substances are not commercially available, the quantification and validation of the determination of the 1,2-difluorochloroethene isomers (HCFC 1122a/1 and HCFC 1122a/2) are performed using 1,1-difluorochloroethene (HCFC 1122)

Test Preparation:

Connect the liquid phase of the sample cylinder (containing 1,1,1,2-tetrafluoroethane) to the gas valve system (loop) of the gas chromatograph (GC). Then evacuate the gas valve system (loop) of the GC including transfer line via a multiway tap. Open the valves for the sample cylinder and fill the loop cautiously with the sample.

Standard Preparation:

The calibration mixtures (containing each compound) are prepared from the pure reference substances (when available) by subsequent dilution in helium.

System Suitability Tests:

The resolution “a” between the peaks of trans-octfluorobutene-2 and cis-octafluorobutene-2 should be greater man 1.4 in the chromatogram of the standard preparation. The tailing factor of 1,1-dichloro-1,2,2,2-tetrafluoroethane should be between 0.8 and 1.2 in the chromatogram of the standard preparation.

### Calculation

The quantitation of the characterized impurities, including those, which are unspecified and summarized in the “sum” or “total” parameters, is performed individually by means of external standard calibrations (when available). If the compound is unavailable, other similar standards are used (see table 1). If the compound is unidentified, the quantitation is performed by external standard calibration with 1,1-difluorochloroethene using the total ion chromatogram.

FIGS. 1-26, 28, 30-41:

Extracted ion chromatograms and mass spectra analyses of spiked 1,1,1,2-tetrafluoroethane/helium gaseous mixtures containing concentrations of about 2 to 6 ppm (v/v) of each compound (when available) listed in table 1.

FIGS. 27 and 29:

Extracted ion chromatograms and mass spectra analyses of a sample of HFC 134a technical grade containing about 5 ppm (v/v) of the 1,2-difluorochloroethene isomer 1 (FIG. 28), and about 0.2 ppm (v/v) of the 1,2-difluorochloroethene isomer 2 (FIG. 30).

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

Validation Data:

Linearity and Range

The linearity of the method was tested by analyzing gaseous mixtures containing increasing amounts of the compounds in helium.

TABLE 1
Results of the regression analysis (1^{st} order)

| | Correlation | Range |
|---|---|---|
| Components | coefficient r | [ppm (v/v)] |
| CFC-13 | 0.9999 | 0.5-16 |
| HFC-23 | 0.9999 | 0.5-20 |
| CFC-115 | 1.0000 | 0.2-16 |
| HFC-143a | 0.9999 | 1-18 |
| HFC-32 | 1.0000 | 0.5-19 |
| HFC-1123 | 0.9999 | 0.5-16 |
| HFC-125 | 0.9999 | 0.5-16 |
| FC-1318my -trans | 1.0000 | 0.2-13 |
| HFC-1234yf | 1.0000 | 0.2-15 |
| HFC-245cb | 1.0000 | 0.5-19 |
| HFC-1225ye (cis) | 1.0000 | 0.5-13 |
| HFC-152a | 0.9999 | 1-19 |
| HFC-161 | 1.0000 | 0.5-16 |
| HFC-1243zf | 0.9999 | 0.5-17 |
| CFC-12 | 0.9999 | 0.5-17 |
| HFC-134 | 1.0000 | 1-140 |
| HFC-1336mzz (cis) | 0.9998 | 0.5-17 |
| HCFC-22 | 0.9997 | 1-20 |
| CFC-114 | 1.0000 | 0.5-15 |
| CFC-114a | 0.9998 | 0.5-15 |
| HCC-40 | 0.9999 | 0.5-13 |
| HCFC-1122 | 0.9998 | 0.5-15 |
| HCFC-124a | 0.9999 | 1-16 |
| HCFC-124 | 0.9999 | 1-15 |
| HCFC-31 | 0.9998 | 1-16 |
| CFC-12B1 | 0.9997 | 1-15 |
| HFC-152 | 0.9998 | 1-17 |
| HCFC-133a | 0.9999 | 1-18 |
| CFC-1112a | 0.9998 | 0.2-16 |
| CFC-11 | 0.9996 | 0.5-18 |
| HCFC-123a | 0.9995 | 1-18 |
| HCFC-123 | 0.9996 | 1-18 |
| CFC-113 | 0.9996 | 1-17 |
| HCFC-1121 (trans) | 0.9995 | 0.5-15 |
| HCFC-132b | 0.9992 | 1-18 |

Accuracy

The accuracy of the method was evaluated by determining the rate of recovery of synthetic gaseous mixtures of the listed components in 1,1,1,2-tetrafluoroethane/helium Three different concentrations (low, medium and high) were prepared in the range considered and tested 3 times. The blank concentrations were considered in the calculation of the recovery rates.

For the components examined, (3 different concentrations in the ranges of approximately 1 to 10 ppm, and approximately 2 to 100 ppm) average recovery rates within a range of 89 to 127% were determined.

Hence, the accuracy of the method was deemed acceptable.

Precision

The precision (repeatability) of the method was evaluated by six determinations of a synthetic gaseous mixture of the compounds in helium at the specification limit.

TABLE 1
Results of precision

| Compound | Relative Standard Deviation % |
|---|---|
| CFC-13 | 2.3 |
| HFC-23 | 1.8 |
| CFC-115 | 2.6 |
| HFC-143a | 2.2 |
| HFC-32 | 2.5 |
| HFC-1123 | 2.5 |
| HFC-125 | 2.4 |
| FC-1318my-trans | 2.7 |
| HFC-1234yf | 2.2 |
| HFC-245cb | 1.8 |
| HFC-1225ye (cis) | 2.2 |
| HFC-152a | 1.5 |
| HFC-161 | 2.5 |
| HFC-1243zf | 1.4 |
| CFC-12 | 1.7 |
| HFC-134 | 1.6 |
| HFC-1336mzz (cis) | 1.7 |
| HCFC-22 | 1.8 |
| CFC-114 | 1.9 |
| CFC-114a | 1.9 |
| HCC-40 | 1.6 |
| HCFC-1122 | 1.7 |
| HCFC-124a | 2.1 |
| HCFC-124 | 1.5 |
| HCFC-31 | 1.5 |
| CFC-12B1 | 1.6 |
| HFC-152 | 1.7 |
| HCFC-133a | 1.9 |
| CFC-1112a | 1.8 |
| CFC-11 | 1.5 |
| HCFC-123a | 1.3 |
| HCFC-123 | 1.4 |
| CFC-113 | 1.6 |
| HCFC-1121 (trans) | 1.9 |
| HCFC-132b | 1.3 |

Limits of Detection and Quantitation

The determination of the limits of detection and quantitation was performed (according to ICH guidelines) during the analyses of linearity. Analyses of gas mixtures of specified compounds having concentrations up to the specification limit were performed. For unspecified compounds, maximum concentrations of approximately 5 ppm were considered.

On the basis of these analyses, the slope “a” together with the residual standard deviation of the regression line was determined according to linear regression. From this the detection and quantitation limits were established. Formula for calculation:
LOD=3.3*σ/a
LOQ=10*σ/a

LOD: Limit of detection

LOQ: Limit of quantitation

σ: Residual standard deviation

a: Slope of the regression straight lines

TABLE 2
Detection and quantitation limits

| | LOD | LOQ |
|---|---|---|
| Compounds | ppm (v/v) | ppm (v/v) |
| CFC-13 | 0.14 | 0.44 |
| HFC-23 | 0.17 | 0.52 |
| CFC-115 | 0.05 | 0.16 |
| HFC-143a | 0.24 | 0.74 |
| HFC-32 | 0.12 | 0.37 |
| HFC-1123 | 0.14 | 0.44 |
| HFC-125 | 0.16 | 0.48 |
| FC-1318my-trans | 0.06 | 0.19 |
| HFC-1234yf | 0.07 | 0.22 |
| HFC-245cb | 0.15 | 0.46 |
| HFC-1225ye (cis) | 0.14 | 0.42 |
| HFC-152a | 0.31 | 0.95 |
| HFC-161 | 0.16 | 0.48 |
| HFC-1243zf | 0.17 | 0.53 |
| CFC-12 | 0.21 | 0.64 |
| HFC-134 | 0.28 | 0.85 |
| HFC-1336mzz (cis) | 0.20 | 0.61 |
| HCFC-22 | 0.35 | 1.06 |
| CFC-114 | 0.17 | 0.50 |
| CFC-114a | 0.17 | 0.51 |
| HCC-40 | 0.10 | 0.31 |
| HCFC-1122 | 0.16 | 0.48 |
| HCFC-124a | 0.27 | 0.80 |
| HCFC-124 | 0.30 | 0.92 |
| HCFC-31 | 0.32 | 0.96 |
| CFC-12B1 | 0.29 | 0.87 |
| HFC-152 | 0.25 | 0.75 |
| HCFC-133a | 0.24 | 0.71 |
| CFC-1112a | 0.06 | 0.19 |
| CFC-11 | 0.14 | 0.41 |
| HCFC-123a | 0.36 | 1.10 |
| HCFC-123 | 0.27 | 0.82 |
| CFC-113 | 0.30 | 0.90 |
| HCFC-1121 (trans) | 0.14 | 0.43 |
| HCFC-132b | 0.35 | 1.07 |

TABLE 3
Comparison of the different GC parameters

| | GC | GC | GC |
|---|---|---|---|
| Features | parameter 1 | parameter 2** | parameter 3** |
| Resolution | 1.66 | 1.68 | 1.67 |
| (FC-1318my-cis/FC- | | | |
| 1318my-trans) | | | |
| Tailing Factor | 0.95 | 0.97 | 0.96 |
| (CFC-114a) | | | |

**Changed method

The changes made to the GC parameters had no significant effect on the separation efficiency of the method or on the peak shape of critical components.

1. Method for the analysis of the content of organic impurities in 1,1,1,2-tetrafluoroethane, in which
(a) the 1,1,1,2-tetrafluoroethane is subjected to a gas chromatography operation and;
(b) an operation is carried out in which the organic impurities are detected by mass spectrometry and wherein said method is carried out using the specific conditions and/or making use of any of the quality control test or validation data included in the specification.

2. Method according to claim 1, in which the initial temperature of the gas chromatography operation is adjusted at the most to 400 C.

3. Method according to claim 1, in which the initial temperature of the chromatography operation is less than or equal to approximately −200 C.

4. Method according to claim 1, in which detection is carried out using the selected ion monitoring (SIM) technique.

5. Method according to claim 1, in which detection is carried out using the time-of-flight (TOF) technique.

6. Process for the manufacture of 1,1,1,2-tetrafluoroethane comprising the use of the method according to claim 1 for controlling the quality of the 1,1,1,2-tetrafluoroethane.

7. Process for the manufacture of a pharmaceutical aerosol, comprising at least one 1,1,1,2-tetrafluoroethane of pharmaceutical grade, comprising the use of the method according to claim 1 for controlling the quality of the 1,1,1,2-tetrafluoroethane of pharmaceutical grade.

8. Method according to claim 3, in which detection is carried out using the selected ion monitoring (SIM) technique.

9. Method according to claim 3, in which detection is carried out using the time-of-flight (TOF) technique.

10. Process for the manufacture of 1,1,1,2-tetrafluoroethane comprising the use of the method according to claim 3 for controlling the quality of the 1,1,1,2-tetrafluoroethane.

11. Process for the manufacture of a pharmaceutical aerosol, comprising at least one 1,1,1,2-tetrafluoroethane of pharmaceutical grade, comprising the use of the method according to claim 3 for controlling the quality of the 1,1,1,2-tetrafluoroethane of pharmaceutical grade.
